(19) United States
(12) Patent Application Publication
(10) Pub. No.: US 2019/0360956 A1
(43) Pub. Date: Nov. 28, 2019

(54) System and Method for Detecting a Section of Lead Piping in a Field Setting

(71) Applicant: Imperia Engineering Partners LLC, Milford, MA (US)

(72) Inventors: Ronald G. Ballinger, Andover, MA (US); Vincent Roy, Millis, MA (US); Agnes Jallouli, Shrewsbury, MA (US)

(21) Appl. No.: 16/421,066

(22) Filed: May 23, 2019

Related U.S. Application Data
(60) Provisional application No. 62/675,723, filed on May 23, 2018.

(51) Int. Cl. G01N 27/04; G01N 33/2028

(52) U.S. Cl. CPC G01N 27/041; G01N 33/2028

(57) ABSTRACT

Embodiments of the present disclosure relate to a method and system for detecting a presence and/or quantity of lead within a section of buried piping, without excavation, by calculating electrical resistance of a section of the piping. At least one embodiment of the present disclosure comprises coupling source leads of wire and sensor leads of wire to separated locations of a section of piping buried in a field setting. After the source leads and sensor leads are coupled, electrical current flow may be delivered to the section of piping via the source leads. A voltage level between the sensor leads may be measured via a voltmeter connected to the sensor leads. Resistance of the section of piping, as a function of the electrical current flow and the measured voltage level, is calculated to determine whether at least a portion of the section of piping includes lead and/or quantity of lead.

### RELATED APPLICATION

This application claims the benefit of U.S. Provisional Application No. 62/675,723, filed on May 23, 2018. The entire teachings of the above application are incorporated herein by reference.

### BACKGROUND

Lead pipes present an important health concern that has attracted national media attention in recent years. Identification of buried lead pipes without any excavation is of utmost importance. Current systems and methods to identify pipe material without excavation exist, but have significant disadvantages.

For example, U.S. Pat. No. 7,125,482 (“the '482 patent”) relates to the material identification of a buried pipe by measuring the electrochemical pipe-to-soil potentials at different locations. The '482 patent describes electrifying the soil and pipes through the groundbed and measuring electrical potential with sensing electrodes. The advantage of this technique is that it is contactless. At least one disadvantage is that it provides only a local measurement of the pipe which is a function of the number and location of the sensing probes. For this technique to be performed, it is important to know the location of the pipe in the ground. Further this technique does not account for the possible interference of nearby utilities or other pipes.

Another method for identifying the material a pipe consists of is Eddy Current Testing. This method relies on a test coil being inserted in a section of pipe (e.g., a water line), and measuring the changes in the impedance of a test coil. AC current is applied to the test coil which creates a magnetic field. This magnetic field induces an eddy current in the surrounding pipe. The eddy current will create a secondary magnetic field that changes the impedance of the test field. The material of the pipe may be identified using this method. However, this method requires inserting the test coil into the pipe which creates issues as it is difficult to navigate through sharp bends, turns, and any stop valves in the pipe. Also, water service is required to be interrupted during measurement.

### SUMMARY

Embodiments of the present disclosure relate to methods and systems for detecting the presence and/or quantity of lead within a section of buried piping by measuring electrical resistance or conductance of the section of piping. Electrical resistivity and electrical conductivity are reciprocals of each other, and one of skill in the art would recognize that embodiments of the present disclosure may be performed based on determining electrical resistance or conductance of piping. For example, a lower conductance value may indicate the presence of lead in the same manner as a higher resistance value.

Lead has a significantly higher resistivity than copper, and it is expected that the resistance of piping with portions of lead will be much higher than if the piping were entirely made of copper. The theoretical value for the resistivity of copper is 1.71×10⁻⁸ Ω/m, and the theoretical value for the resistivity of lead is 21.3×10⁻⁸ Ω/m. Thus, the ratio of the resistivity of lead to copper is 12.5.

However, in practice, the resistance of copper and lead pipes buried underground may be different. For example, copper and lead pipes removed from the field have resistivity measured at 2.13×10⁻⁸ Ω/m for copper, and 14.3×10⁻⁸ Ω/m for lead. While the resistivity of copper increased over its theoretical value and the resistivity of lead decreased from its theoretical value, the two resistivity values are still different by a ratio of 7. This difference in the resistivity values of copper and lead is enough to be able to measure a significant difference of resistance when lead is present in a buried section of piping.

In addition to identifying the difference between the theoretical and actual resistivity values for copper and lead piping, experimental testing demonstrated that the conditions of the buried piping, such as oxide on the surface, dirt, cracks, and holes, do not significantly affect the resistance. The resistance measurement can also be reliably performed with water inside the piping. Further, the surrounding soil will not significantly affect the resistance measurement either. Further still, the effect of a pipe coupling, that may or may not be present in a section of piping, such as a Copper-Copper (Cu—Cu) coupling or a Copper-Lead (Cu—Pb) coupling, was also considered. A Cu—Cu coupling does not change the value of the resistivity. In contrast, a Cu—Pb coupling increases the measured resistance. The amount of the increase depends on the type of coupling. The values vary from an increase of 6.5 μΩ to open.

Embodiments of the present disclosure include a method and apparatus capable of determining significance of electrical resistance of a section of piping, and how the value of the electrical resistance relates to the presence and/or quantity of lead in the section of piping. The term “piping” is inclusive of pipe, and other parts or features of a pipe system (e.g., a curb stop, a valve, a pipe coupling, a water meter, a water spigot, etc.).

At least one embodiment of the present disclosure comprises coupling source leads of wire to first separated locations of a section of piping buried in a field setting, and coupling sensor leads of wire to second separated locations of the section of piping, wherein the second separated locations of the section of piping are located between the first separated locations. After the source leads and sensor leads are coupled to the piping, electrical current flow may be delivered to the section of piping via the source leads. The voltage level between the sensor leads may be measured via a voltmeter connected to the sensor leads. In some embodiments, the distance between the sensor leads of wire (i.e., the distance between the second separated locations) is measured. In some scenarios it may be prohibitively difficult to measure the distance between the sensor leads, in such cases the distance may be estimated. The resistance of the section of piping, as a function of the electrical current flow and the voltage level, is calculated to determine whether at least a portion of the section of piping includes lead content as a function of the calculated resistance and the measured distance.

According to some embodiments, the determination of whether at least a portion of the section of piping includes lead content may be a function of the calculated resistance, measured distance (or estimated distance) between the sensor leads, an inside diameter of the section of piping, and an outside diameter of the section piping. Some embodiments further include using probability detection techniques to determine a probability that the determination of whether at least a portion of the section of piping includes lead content is correct. Some embodiments further include determining a length of the portion of the section of piping that includes lead content, the length of the portion of piping being calculated as a function of the calculated resistance, the measured distance (or estimated distance) between the sensor leads, an inside diameter of the section of piping, an outside diameter of the section of piping. In some embodiments, the determination of whether at least a portion of the section of piping includes lead content is a function of the calculated resistance, the measured distance, resistivity of copper, and resistivity of lead.

According to some embodiments, the electrical current flow delivered to the section of piping via the source leads may be varied or adjusted depending on the amount of resistance being measured. In some embodiments, varying the electrical current flow delivered to the section of piping via the source leads comprises delivering multiple different amounts of electrical current. For example, the electrical current flow delivered to the section of piping via the source leads may be varied by delivering 10 amperes of electrical current, and delivering 1 ampere of electrical current. In some embodiments, varying the electrical current flow delivered to the section of piping via the source leads comprises delivering electrical current flow in a first direction, and delivering electrical current flow in a second direction.

Some embodiments include a first source lead and a second source lead, and a first sensor lead and a second sensor lead. In some embodiments, the first source lead and the first sensor lead are coupled to locations of the section of piping that are separated by at least 1.5 times the cross-sectional perimeter of piping, and the second source lead and the second sensor lead are coupled to locations of the section of piping that are separated by at least 1.5 times the cross-sectional perimeter of the piping.

Some embodiments may include determining whether at least a portion of the section of piping includes lead content as a function of the measured voltage level without explicitly calculating a resistance. For example, the measured voltage may be compared to pre-calculated voltages in a table for particular electrical currents and physical characteristics of piping (e.g., lengths, inside diameters, outside diameters) to determine whether at least a portion of the section of piping includes lead content. Additionally, one of skill in the art would understand that, equivalent to calculating the resistance of the section of piping as a function of the electrical current flow and the voltage level, one could derive an equation to determine whether at least a portion of the section of piping includes lead content as a function of the measured voltage level instead of resistance because voltage is equal to current times resistance. Thus, the measured voltage for a particular current is mathematically interchangeable with the respective resistance.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a table of calculated values of the electrical resistance of water lines with combinations of lengths of copper and lead pipes.

FIG. 2 is a table of descriptions of various pipe samples removed from the field for testing.

FIG. 3 is a table of the results of experimental conductivity testing of the pipe samples.

FIG. 4 is a table of the results of experimental conductivity testing of the pipe samples where the connection points for the leads were cleaned.

FIG. 5 is a table of the results of experimental conductivity testing of the pipe samples with and without water.

FIG. 6 is a table of the resistances of pipe couplings.

FIG. 7 illustrates an embodiment of the present disclosure for detecting presence of lead in piping in a field setting.

FIG. 8 is a graph of the value of the resistance of a section of pipe as a function of lead content.

FIG. 9 is a block flow diagram of an example method for detecting a section of lead in piping in a field setting.

FIG. 10 is a block flow diagram of example instructions executed by a lead detection module.

FIG. 11 is a diagram of the internal structure of a computer.

### DETAILED DESCRIPTION

A description of example embodiments follows.

Embodiments of the present disclosure provide for detecting the presence and/or quantity of lead within a section of buried piping without the drawbacks of prior art as discussed above. Embodiments of the present disclosure avoid the need to excavate a buried section of pipe unnecessarily by measuring the electrical resistance of the section of piping to determine if a portion of the section of piping is or contains lead.

One of skill in the art would understand that embodiments of the present disclosure may be used to detect the presence of lead in a section of piping in many different applications. One example problem that embodiments of the present disclosure is able to address, without the drawbacks of the prior art, is where the material of a residential water service line is unknown. A residential water service line is the piping that connects a house to a water main that supplies an area with water.

Typically, the water main is buried beneath a street adjacent to the house. For each house that the water main serves, a water service line stems from the water main and runs beneath the residential property (e.g., under the front yard) to each respective home at a point in the foundation. Water service line pipe is typically ¾ to 2 inches in diameter and is buried at a depth determined by the local building code, with colder climates requiring deeper burial.

Generally, the water utility company or the municipality that supplies the water is responsible for the maintenance of the water main, but the homeowner is responsible for the water service line that runs beneath their property. With each individual water service line being the responsibility of the home owner, it is often unknown if the water service line pipe or piping serving a home is made of lead or copper. Lead pipes present an important health concern, and homeowners, as well as prospective home purchasers, wish to know if the water service line to the home contains any lead pipe. However, accessing the buried water service line is costly and difficult, and current methods of material identification of buried pipe have the drawbacks described above.

To solve this issue without the drawbacks of the prior art, a method and system for detecting a section of lead pipe in a field setting by measuring the electrical resistance of the section of piping was designed and developed. Described below is experimental testing and development that proved the effectiveness of this concept, and integrated the concept into a practical application suitable for use in the field.

### Measurement Approach

The resistance of a water service line piping made of lead and copper parts may be modeled by the following formula:

$$R = {R}_{Cu} + {R}_{Pb} = {[ {ρ}_{Cu} \frac{{L}_{Cu}}{{S}_{Cu}} ]}_{Cu} + {[ {ρ}_{Pb} \frac{{L}_{Pb}}{{S}_{Pb}} ]}_{Pb}$$

Where: R_{Cu} is the resistance of the copper portion of the water line,
   R_{Pb} is the resistance of the lead portion of the water line,
   ρ_{Cu} and ρ_{Pb} are the resistivity of copper and lead, respectively,
   L_{Cu} is the length of the copper portion of the water line,
   L_{Pb} is the length of the lead portion of the water line,
   S_{Cu} and S_{Pb} are the surface area of the metal for each material.
With S=0.25 π(OD²−ID²); OD: external diameter, ID: internal diameter; and
   Total length of the water line=L_{Cu}+L_{Pb}.

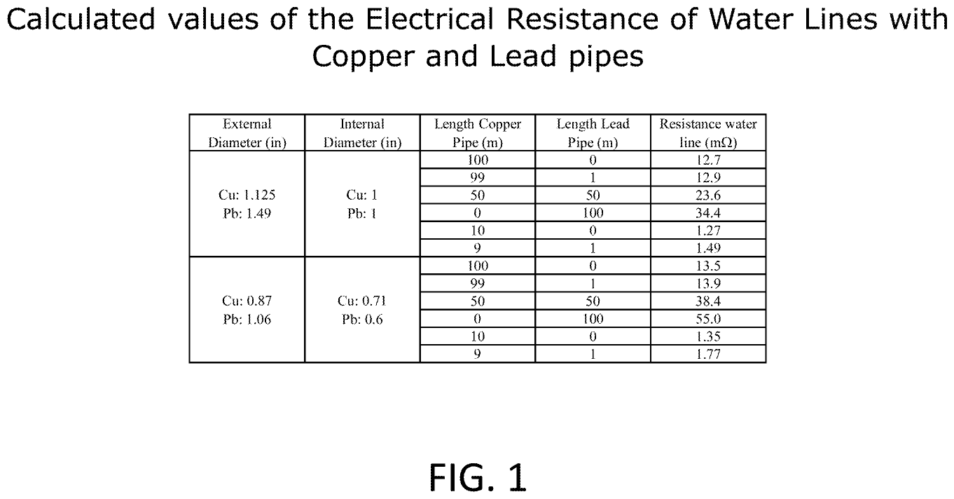

This formula is an approximation as it does not take into account an impact of a connection between the lead and copper pipes. Examples of calculated values for water line piping with a mix of copper and lead pipes are presented in the table of FIG. 1.

FIG. 1 is a table of calculated values of the electrical resistance of water lines with combinations of lengths of copper and lead pipes. As shown in the table of FIG. 1, the theoretical value of the resistance of a 100-meter water pipe will be changed even if only 1 meter of lead is present. The change in the resistance value is in the order of a 1/10 of a mat. This change can be measured with a micro-range ohmmeter.

### Laboratory Testing

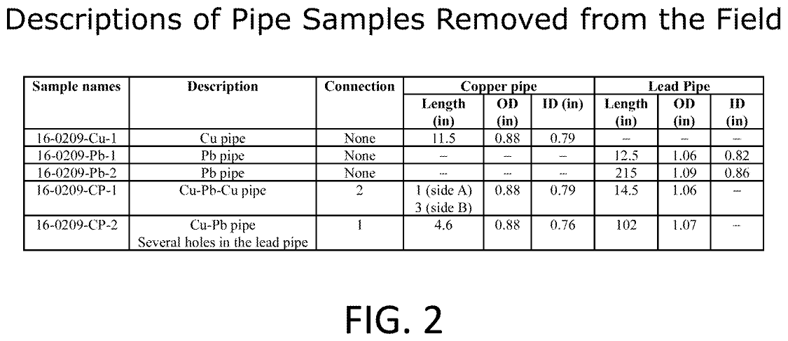

Laboratory testing was performed to prove the concept that the resistance of piping in the field can be used to determine the presence of lead. Several samples of pipe were removed from the field for evaluation. The pipe samples are actual water service line pipes removed from supply service for residential homes. These samples are listed and described in the table of FIG. 2. Each of the samples was measured at least four times, and the measurements were averaged together to determine the sample's dimensions. Overall, the averages obtained for each sample showed that the pipes sampled had the following dimensions:

Copper Pipe

| | |
|---|---|
| OD (in) | 0.88 |
| ID (in) | 0.78 |

Lead Pipe

| | |
|---|---|
| OD (in) | 1.07 |
| ID (in) | 0.84 |

### The Measurements of Pipe Resistance

A Low Resistance Ohmmeter (LRO) was used to measure the resistance of each sample pipe. The LRO chosen uses four lead wires to perform a low resistance measurement. Two leads are used for providing a current (C1 and C2), also referred to herein as source leads, through the sample pipe, and two other leads (P1 and P2), also referred to herein as sensor leads, are used to measure the potential (i.e., voltage) on the sample pipe. The potential leads were placed on the inside between the current leads. Before any resistance was measured, two points on each sample pipe were chosen for where to apply the sensor leads. The distance between those two points was then measured.

Leads were applied on each sample pipe with the position of leads C1 and C2 on the outside, and leads P1 and P2 placed in-between C1 and C2. A solid connection of both leads was made simultaneously to create a full circuit. The LRO was first set to provide 10 A of current, which, in this case was the most sensitive setting. If the resistance was undetectable because it was out of the range, the LRO was then set to 1 A for a lower current. The resistance measurement was repeated three times for accuracy purposes.

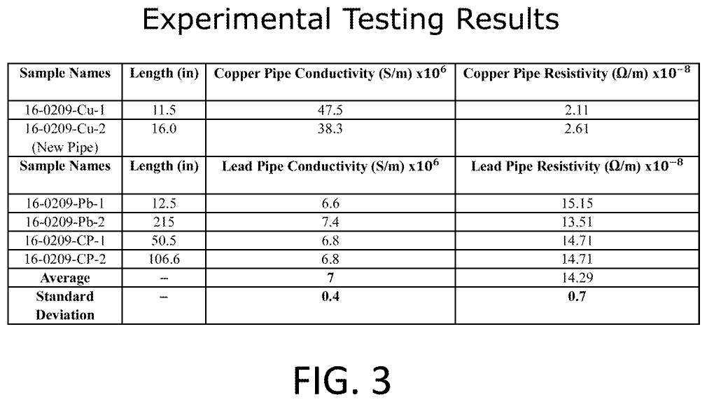

The conductivity of the samples from the field were measured. The results obtained are shown in the table of FIG. 3. No cleaning or other preparation was done on the pipes prior to measuring the resistance. From the field samples received for analysis, only one copper section was long enough to measure the resistance (16-0209-Cu-1). The conductivity for a copper pipe was found to be 47.5×10⁶ siemens per meter (S/m), with a resistivity of 2.11×10⁻⁸ Ω/m. Resistivity is a measure of the resisting power of a material to the flow of an electric current. The SI unit of electrical resistivity is the ohm-meter (Ω·m). Conductivity is the reciprocal of electrical resistivity, and represents a material's ability to conduct electric current. The SI unit of electrical conductivity is siemens per meter (S/m).

Several samples were used to determine the conductivity/resistivity of lead pipes. The average of the measurements resulted in a conductivity of 7×10⁶ S/m and a resistivity of 14.3×10⁻⁸ Ω/m for the lead pipes.

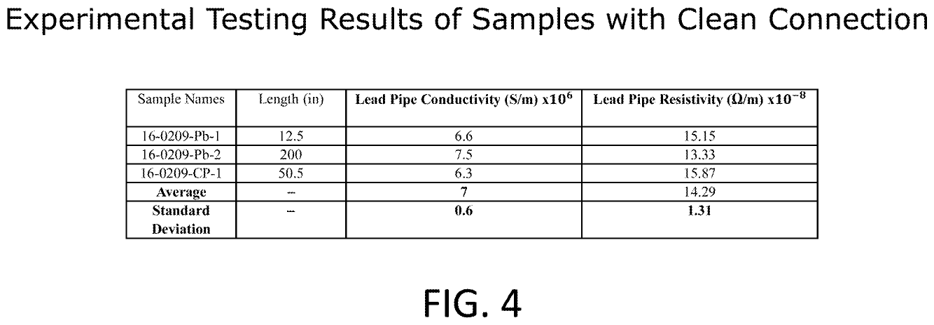

The results obtained for the lead pipes had some dispersion. To test if this variability was due to a poor connection of the leads to the pipes, the area where the leads were applied was removed of oxides by cleaning with sand paper, and the measurements were made again. The results from retesting of the pipes with a clean connection are shown in the table of FIG. 4.

Conductivity values obtained with a clean connection (shown in the table of FIG. 4) are similar to those obtained without cleaning the surface of the pipe (shown in the table of FIG. 3). Thus, the removal of oxides on the surface did not significantly affect the test results.

The data shows that the copper pipes have a lower experimental conductivity (47.5×10⁶ S/m) than theoretical conductivity (58.5×10⁶ S/m). In contrast, the lead pipes' experimental conductivity values (7×10⁶ S/m) are much higher than what is expected based off the theoretical conductivity value of 4.7×10⁶ S/m. However, the conductivity of copper is still larger than the conductivity of lead by a factor of nearly 7.

As described above, pipes removed from the field were not cleaned prior to performing testing. Thus, dirt and oxidation were present on the pipes. The cleanest sample was 16-0209-Pb-1, and the dirtiest sample was 16-0209-CP-2. There were no significant differences in the conductivity value obtained between those two samples, as shown in the table of FIG. 3. Also, sample 16-0209-CP-2 had several holes and cracks. The conductivity value obtained for 16-0209-CP-2 (see the table of FIG. 3) shows that there is no difference in the value compared to the results obtained with the other sample lead pipes. Thus, the presence of pipe defects, such as an irregular surface, holes or cracks have no influence on the conductivity/resistivity measurement.

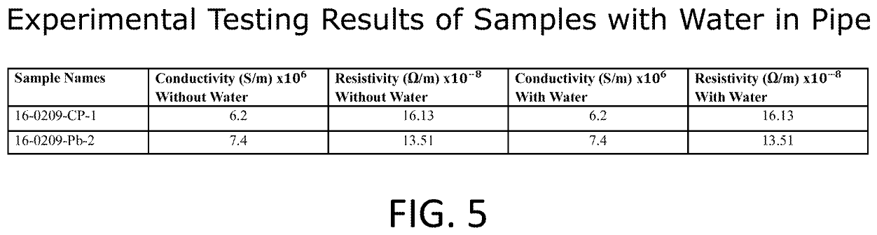

In the field, the pipe is buried in the soil and filled with tap water. Thus, measurement of the resistance/conductance of sample pipes filled with tap water was performed. The comparative resistivity/conductivity measured for the empty and filled sample pipe are shown in the table of FIG. 5. The results in the table of FIG. 5 show that the presence of water in the pipes does not interfere with the conductivity measurements. Tap water has a much higher conductivity than soil, so it should be more susceptible to interfere with the measurement than soil. From the results obtained with the pipe filled with water, it is concluded that soil would also have minimal influence on the resistivity/conductivity measurement.

Two coupling types were evaluated for the sample pipes received from the field. First a Cu—Cu coupling, and secondly, a Cu—Pb coupling. In the samples received, there were three different configurations of Cu—Pb couplings. Resistance was measured including the copper side, the coupling and, the lead side to determine the contribution of the Cu—Pb coupling. The resistance R measured can be expressed as three contributions:

R=R_{Cu}+R_{Pb}+R_{Cu—Pb}
Where R_{Cu} is the resistance of the Cu pipe
   R_{Pb} is the resistance of the Pb pipe
   R_{Cu—Pb} is the resistance of the coupling Cu—Pb.

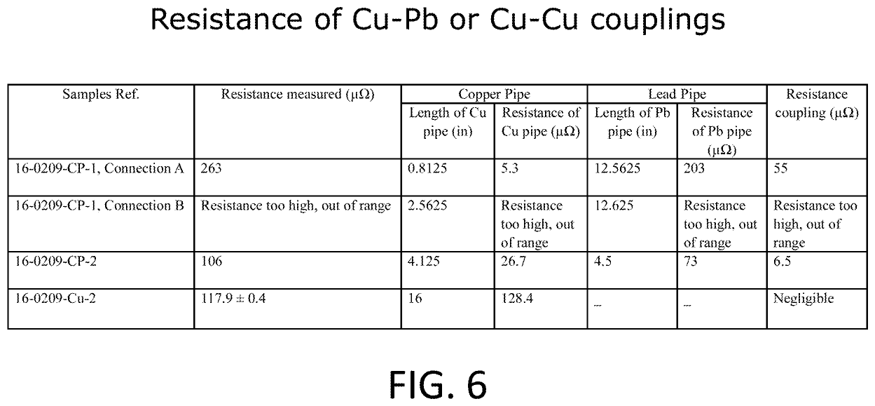

The resistance of the coupling was calculated by subtracting the portion of the resistance due to the copper pipe and the lead pipe calculated from the resistivity of copper and lead and the respective length of each pipe. The results obtained are presented in the table of FIG. 6.

Based on these results, the connection between copper and lead pipe will increase the resistance. In one type of connection (16-0209-CP-1, Connection B), the resistance was too high to be measured with a LRO. The lowest value connection measured was a resistance of 6.5 μΩ for 16-0209-CP-2.

For a Cu—Cu connection, the resistance of the pipe with the connection was comparable to a similar length copper pipe without the Cu—Cu connection. It may be concluded that there is not significant contribution of the Cu—Cu connection to the overall resistance of the pipe.

Based in part on the knowledge gained from the laboratory testing, a lead detection technique was developed to help assist in the interpretation of field measurements. In a postulated field scenario, a resistance measurement would be performed between the house (at copper piping inside home or other accessible location) and the street water shutoff. The lead detection device both performs the resistance measurement between those two locations and aids in its interpretation to determine the presence of lead.

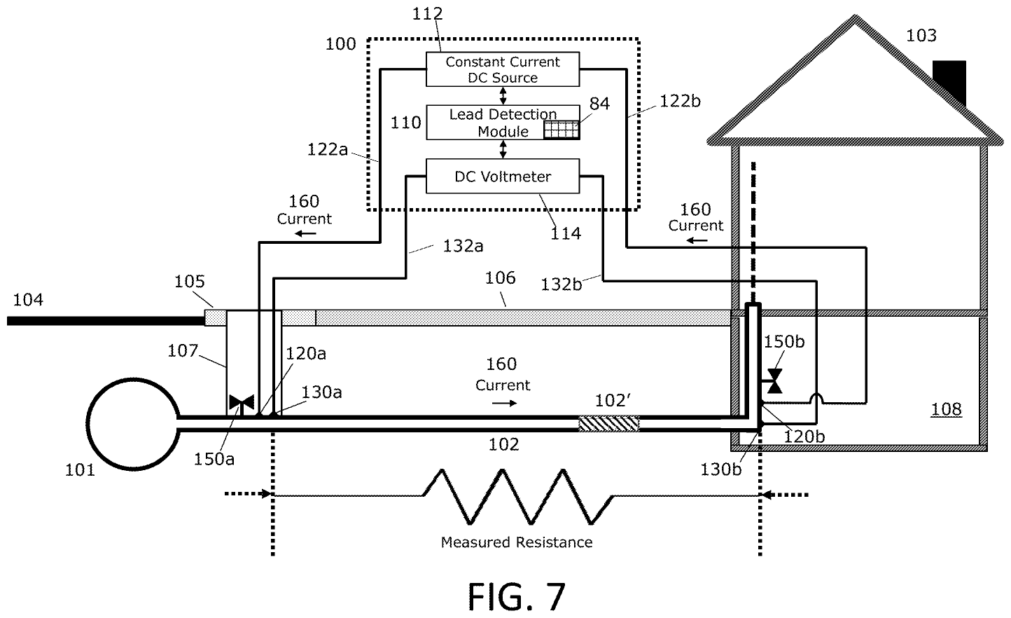

One example scenario depicted in FIG. 7 is where homeowners may wish to determine if the water service line that supplies their home with water contains lead. One of skill in the art would understand that FIG. 7 illustrates a non-limiting example scenario, and that embodiments of the present disclosure may be used to detect the presence of lead in many different applications.

FIG. 7 illustrates an example embodiment for detecting the presence of lead in a water service line piping 102 between a water main 101 and a home 103. In this example scenario, a water main 101 (depicted as running into the page and perpendicular to the water service line 102) owned by a water utility company supplies water to a home 103 via a water service line 102.

Generally, the water utility company is responsible for the water main 101 and the section of the water service line piping 102 between the water main 101 and the street water valve 150a. The homeowner is generally responsible for water service line 102 after the street water valve 150a. This may create a situation where the homeowner or a prospective buyer does not know whether the section of the water service line piping 102 running from the street shutoff valve 150a, under the sidewalk 105 and front yard 106, to the basement 108 of the home 103 contains any lead pipe or other lead parts of a pipe system.

Further, while the example below is described from the perspective of the home owner's water service line 102, one of skill in the art would recognize that embodiments of the present disclosure would be applicable to other use cases involving piping. For example, the embodiments describe below are also capable of detecting the presence of lead in the section of piping between the water main 101 and the street shutoff valve 150a. Further still, one of skill in the art would understand embodiments of the present invention may be used on other products/materials besides piping to determine the presence of lead.

An example embodiment of the present disclosure is a lead detection device 100 that is capable of detecting the presence of lead in the water service line 102 in part by calculating the resistivity of the water service line piping 102. The lead detection device 100 takes advantage of the important difference of resistivity between copper and lead. Copper's theoretical resistivity is 1.71×10⁻⁸ Ω/m while lead's theoretical resistivity is 21.3×10⁻⁸ Ω/m, a ratio of 12.5. However, copper and lead pipes that are used as water service lines are expected to have a resistivity of approximately 2.13×10⁻⁸ Ω/m and 14.3×10⁻⁸ Ω/m, respectively. This difference is big enough that measurement of the resistance of pipes will be affected even by the presence of a small amount of lead in a mostly copper water service line piping.

The resistance of the water service line piping 102 will be below 1Ω. Thus, a Low Resistance Ohmmeter or low resistance measurement techniques will be needed to measure its resistivity. Low resistance measurement is used to identify issues in a variety of components and industries.

The lead detection device 100 uses four lead wires 122a, 122b, 132a, and 132b to perform the low resistance measurement of a section of the water service line 102, which includes a portion 102′ composed of lead. Two source lead wires 122a, 122b are used to apply current, and two sensor lead wires 132a, 132b are used for potential measurements. Before any resistance is measured, four points (i.e., two separated sections) on the water service line piping 102 are chosen as locations at which to apply the leads. In this example, the first source lead 122a is coupled at location 120a, near to the street water shut off valve 150a, which is accessed via an access box 107 under the sidewalk 105 in front of the house 103. The second source lead 122b is coupled at location 120b, near the home water shut off valve 150b, which is accessed in the basement 108 of the house 103.

In other situations, the sensor lead wires and the source lead wires may be coupled to a part of the pipe system if the pipe itself is not as accessible as shown in the example above. For example, the lead wires, on one or both ends of the section of piping, may be coupled to a curb stop, a pipe coupling, a water meter, or a water spigot that is part of the section of piping. In such situations, the lead detection device 100 operates in the same manner as if the leads were attached to the pipe itself.

The potential (i.e., voltage) sensor leads 132a and 132b are attached to the water service line 102 in separated locations 130a and 130b, respectively, in-between the current source leads locations 120a and 120b. In order to measure the resistance of the section of the water service line 102 in-between the coupling locations 130a and 130b of the potential sensor leads, the lead detection device 100 may utilize a constant current DC source 112 to provide a constant current 160 through the section of water service line 102. Further, the lead detection device 100 may utilize a DC voltmeter 114 to measure the voltage of the section of the water service line 102 between the coupling locations 130a, 130b of the two potential sensor leads 132a, 132b.

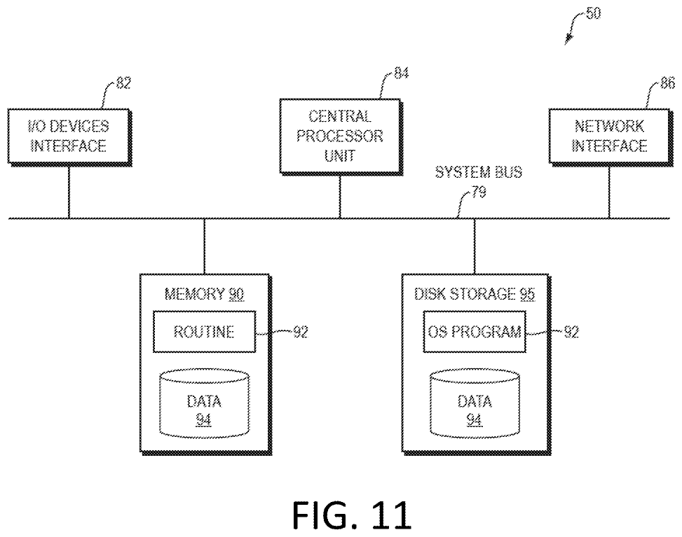

The constant current DC source 112 and the DC voltmeter 114 provide the current and the measured voltage, respectively, to a lead detection module 110. According to some embodiments, the lead detection module 110 calculates the resistance of the section of the water line 102 as a function of the electrical current and the measured voltage (i.e., Resistance=Voltage/Current). In some embodiments, the lead detection module 110 may be implemented as a computer system (e.g., the computer system 50 as shown in FIG. 11 and described below) including a processor 84.

According to the example embodiment depicted in FIG. 7, four lead wires are used to measure the resistance; however, some embodiments may only use two lead wires. The pairs of source and potential leads (i.e., 122a and 132a; 122b and 132b) may be separated or incorporated into a single lead each. If the pairs of leads are separated as shown in FIG. 7, the distance between the coupling locations 120a and 130a of the source lead 122a and potential lead 132a, respectively, and the coupling locations 120b and 130b of the source lead 122b and potential lead 132b, respectively, is preferred to be at least 1.5 times the cross-sectional perimeter of the water service line piping 102 being measured. The potential leads 132a, 132b are placed on the inside of the source leads 122a, 122b. The advantage of the four lead wires is that no current flows into the potential leads 132a, 132b, so the resistance of the potential leads 132a, 132b, as well as their contact resistance, are not measured.

According to some embodiments, the lead detection module 100 lay be configured to eliminate errors created by thermal EMF by causing the constant current DC source 112 to reverse the direction of the current flow and performing the resistance measurement in forward and reverse polarity. In this embodiment, the DC voltmeter 114 provides the two resulting voltage measurements to the lead detection module 110, and the lead detection module 110 is configured to average the absolute value of the voltages together, and use the average to determine the resistance of the section of the water service line 102.

In some embodiments, the lead detection device 100 may be configured to perform the resistance measurement numerous times to increase accuracy. The lead detection device 100 may be configured to calculate the resistance numerous times and average the resistance values together. Each time the resistance is calculated, the calculated resistance may be based on different amounts of current and/or separate voltage measurements.

According to some embodiments, the lead detection device 100 may be configured to adjust the amperage of the current provided by the constant current DC source 112 depending on the amount of resistance being measured. For example, in the micro-ohmmeter range, the constant current DC source 112 may provide 10 A or more. In some embodiments, the lead detection device 100 may be configured to calculate resistance of a section of piping with different amounts of current being provided between 10 A and 1 A current, or in some cases currents less than 1 A. The different amounts of electrical current applied may depend on the length of lead present in the section of piping. For example, if a section of piping consists of more than 7 ft of lead, a 1 A current or less may be needed to detect the resistance. One of skill in the art would understand that the present disclosure is not limited to any specific amount of current in order to calculate resistance from the measured voltage level.

According to some embodiments, the lead detection device 100 may be configured to first provide a 10 A current via the constant current DC source 112, the most sensitive setting. If the resistance was undetectable because it was out of the range, the lead detection device 100 may be configured to then provide 1 A current via the constant current DC source 112.

Some embodiments of the lead detection device 100 may incorporate other methods of low resistance measurement known in the art. Further, the surface of the water service line 102 where the leads 122a, 122b, 132a, and 132b are attached is preferably prepped before the leads are attached to ensure a good connection to the metal of the water service line 102. Further, the leads 122a, 122b, 132a, and 132b may also be attached to the inside surface of the piping.

Additionally, the locations 120a, 130a, 120b, 130b that the source leads 122a, 122b and potential leads 132a, 132b are attached to the water service line piping 102 may vary depending on the application. In this example scenario, the access box 107 in the sidewalk 105 and the basement 108 of the house were the most convenient and accessible.

According to some embodiments, the lead detection module 110 may be configured to receive a representation of a distance between the sensor leads 122a, 122b (i.e., an estimated or measured length of piping between the sensor leads) of the section of the water service line 102. In some cases, it may not be practical to make an accurate measurement of the distance between the sensor leads 122a, 122b, thus, the distance used by the lead detection module 110 may be an estimate.

Further, as shown above, the resistance of a portion of piping is based on the surface area of the metal for each material (i.e., lead and copper) comprising the piping; calculated as S=0.25π(OD²−ID²). Again, in some cases, the exact inner and outer diameter of the piping may not be known or measurable. Thus, the inner and outer diameters used by the lead detection module 110 may be an estimate. In some embodiments, the lead detection module 110 may store default values for inner and outer diameters based on the application of the piping, and use these stored default values to determine the presence of lead in the piping.

In some embodiments, the lead detection module 110 may be configured to determine whether at least a portion of the section of the water service line 102 includes lead content as a function of the measured resistance and length of the section of the water service line 102.

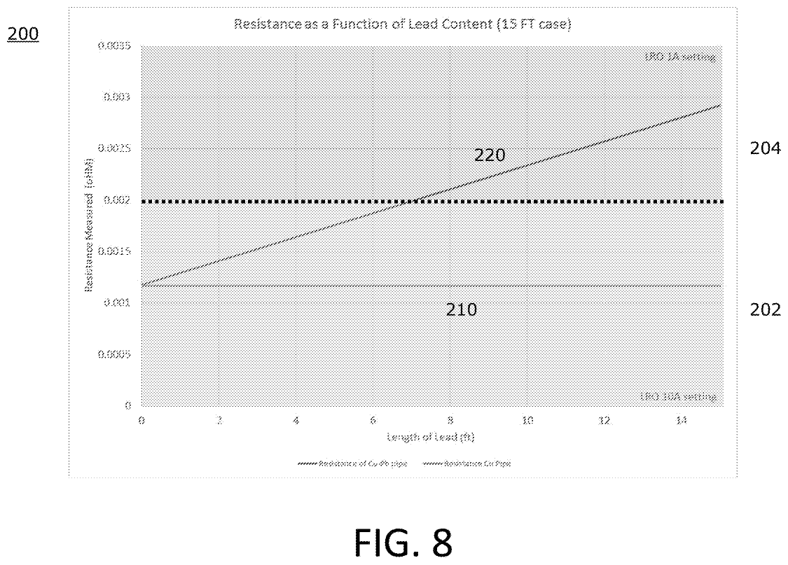

FIG. 8 is a graph 200 of the value of the resistance of an example 15 ft section of piping with various lengths of lead piping. Line 210 represents the resistance of the 15 ft section of piping, if the entire section of piping were made of copper. Line 220 represents the resistance of the 15 ft section of piping as of function of its lead content. In other words, line 220 is a graph of the resistances for all the possible Cu—Pb combinations of the example 15 ft section of pipe. Thus, once the resistance of the section of piping is measured, the measured resistance may be used to determine length of the lead piping (if any) in the section of piping. For example, if the total length of the section of piping is 15 ft (i.e., the distance between the sensor leads), and the calculated resistivity of the section of piping is just less than 2 micro ohms, then approximately 6 ft of the section of piping is lead with the remaining 9 ft being copper.

The graph 200 comprises an upper section 204 in which resistance would be measured with 1 A of current, and lower section 202 in which resistance would be measured with 10 A of current. A section of piping can be measured with either a 10 A current or a 1 A current depending on the length of lead present in the section of piping. If a section of piping consists of more than 7 ft of lead, a 1 A current may be needed to detect the resistance. This calculation demonstrates that for such a length (15 feet) of piping, the presence of lead will always be detectable. This will be the case for a total piping length up to 100 feet. This demonstrates that the method and system of the present disclosure is sensitive enough to always detect lead.

One of skill in the art would understand graphs of various lengths of section of pipe may be derived or calculated by the lead detection module 110, or similar graphs may be derived by the lead detection module 110 based on the received length of the section of the piping.

In some embodiments, the lead detection module may be configured to solve for the amount of lead in a section of pipe of any length using the formula disclosed above in paragraph [0033].

In some embodiments, the lead detection module 110 may also be configured to receive an inside diameter and outside diameter of the piping and determine the surface area of the piping based on the formula S=0.25 π(OD²−ID²), as described above in paragraph [0033].

In other embodiments, the lead detection module 110 may be configured with a stored default value for the inside diameter and outside diameter of the piping (e.g., the values disclosed in paragraph [0036]) or a stored default value for different types of piping (e.g., a residential water service line) based on the average size of the respective type of piping. The lead detection module 110 may be configured to calculate the resistance of the section of piping and solve for the R_{Cu} and R_{Pb} using the formula

$$R = {R}_{Cu} + {R}_{Pb} = {[ {ρ}_{Cu} \frac{{L}_{Cu}}{{S}_{Cu}} ]}_{Cu} + {[ {ρ}_{Pb} \frac{{L}_{Pb}}{{S}_{Pb}} ]}_{Pb} .$$

After solving for the R_{Cu} and R_{Pb}, the lead detection module 110 may be configured to solve for the L_{Cu} and L_{Pb}. The lead detection module 110 may then provide the length of the portion of piping consisting of lead (L_{Pb}) to a user.

According to some embodiments, the lead detection module 110 is capable of determining the significance of the electrical resistance of a section of piping using a simulation-based probability of detection (POD) algorithm. It is known from the state of the art of nondestructive testing (NDT) that NDT techniques are useful for detection of flaws. The purpose of the simulation-based POD algorithm is to determine the probability that the result obtained is true. The result obtained in this specific case will be the presence and length of lead piping in an inspected water distribution system. The simulation-based POD will take into account parameters that can induce false or uncertain results. Such parameters include, but are not limited to: pipe dimensions, pipe material (if different from copper or lead), coupling between two types of pipes, ground loops, or soil/structure interactions.

In some embodiments, the lead detection module 110 is configured to determine the probability that the determination of whether at least a portion of the section of piping includes lead content is correct.

The lead detection module 110 is depicted in FIG. 7 as being located in the lead detection device 100. However, this is only one example embodiment. One of skill in the art would understand that the lead detection module 110 may be located in a remote location. In some embodiments, the lead detection module 110 may control the constant current DC source 112, and receive voltage measurements from the DC voltmeter 114 via a network connection. For example, the lead detection module may be implemented in the cloud or in a software as a service architecture.

The embodiments of the present disclosure described above are capable of determining if lead present in a section of piping by measuring the resistance of the piping. The measurement will not be affected by the presence of oxide or dirt on or inside the piping. The presence of pipe defects, such as an irregular surface, holes or cracks will also have no influence on the measurement. The presence of a Cu—Pb connection will have minimal influence on resistance measured. The environment of the pipe, with water inside and soil outside will have no influence on the value of the resistance.

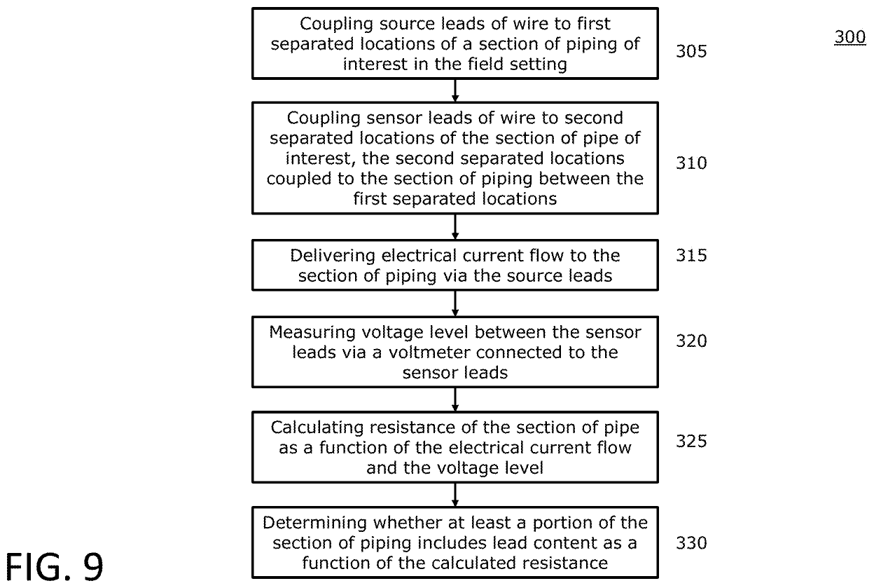

FIG. 9 is a block flow diagram of an example embodiment method 300 for detecting a section of lead in piping in a field setting. According to some embodiments, the method 300 comprises coupling source leads of wire to first separated locations of a section of piping buried in a field setting 305, and coupling sensor leads of wire to second separated locations of the section of piping, wherein the second separated locations of the section of piping are located between the first separated locations 310. After the source leads and sensor leads are coupled to the piping, delivering electrical current flow to the section of piping via the source leads 315. The voltage level between the sensor leads may be measured via a voltmeter connected to the sensor leads 320. The resistance of the section of pipe, as a function of the electrical current flow and the voltage level 325, is calculated to determine whether at least a portion of the section of piping includes lead content as a function of the measured resistance 330.

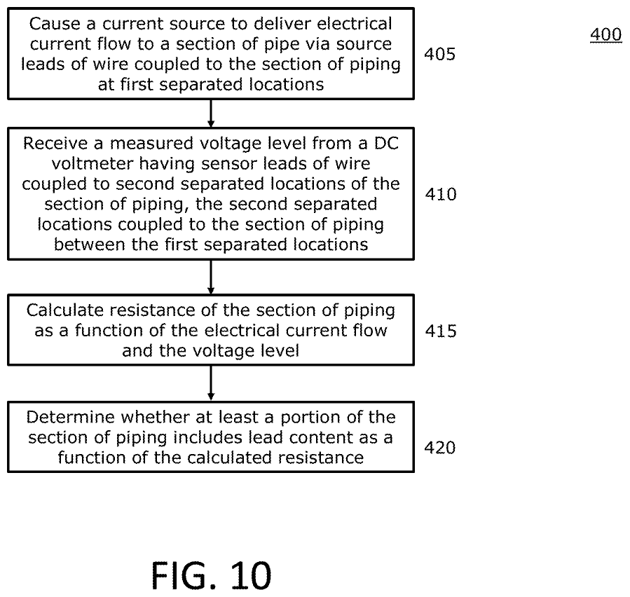

FIG. 10 is a block flow diagram of example instructions 400 executed by a lead detection module 110. According to some embodiments, the lead detection module 110 may be configured to cause a current source to deliver electrical current flow to a section of pipe via source leads of wire coupled to the section of piping at first separated locations 405. The lead detection module may be further configured to receive a measured voltage level from a DC voltmeter having sensor leads of wire coupled to second separated locations of the section of piping, the second separated locations coupled to the section of piping between the first separated locations 410. The lead detection module 110 may be further configured to calculate resistance of the section of piping as a function of the electrical current flow and the voltage level 415 and determine whether at least a portion of the section of piping includes lead content as a function of the calculated resistance 420.

FIG. 11 is a diagram of the internal structure of a computer (e.g., client processor/device 50) in which the lead detection module 110 may be implemented. Each computer 50 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., constant current DC source 112, DC voltmeter 114, keyboard, mouse, displays, printers, speakers, etc.) to the computer 50 (e.g. lead detection module 110). Network interface 86 allows the computer to connect to various other devices attached to a network. Memory 90 provides volatile storage for computer software instructions 92 and data 94 used to implement an embodiment of the present invention (e.g., supporting code to perform methods 300 and 400, etc.). Disk storage 95 provides non-volatile storage for computer software instructions 92 and data 94 used to implement an embodiment of the present invention (e.g., supporting code to perform methods 300 and 400, etc.). Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A lead detection system comprising a non-transitory computer-readable medium including computer code instructions stored therein, the computer code instructions, when executed by at least one processor, enable the lead detection system to:
cause a current source to deliver electrical current flow to a section of piping via source leads of wire coupled to the section of piping at first separated locations;
receive a measured voltage level from a DC voltmeter having sensor leads of wire coupled to second separated locations of the section of piping, the second separated locations coupled to the section of piping between the first separated locations;
calculate resistance of the section of piping as a function of the electrical current flow and the voltage level; and
determine whether at least a portion of the section of piping includes lead content as a function of the calculated resistance.

2. The lead detection system of claim 1, wherein the computer code instructions, when executed by at least one processor, further enable the lead detection system to receive a representation of a distance between the first separated locations; and wherein the determination of whether at least a portion of the section of piping includes lead content is a function of the calculated resistance and the representation of the distance.

3. The lead detection system of claim 2, wherein the determination of whether at least a portion of the section of piping includes lead content is a function of the calculated resistance, the representation of the distance, an inside diameter of the section of piping, and an outside diameter of the section of piping.

4. The lead detection system of claim 2, wherein the computer code instructions, when executed by at least one processor, further cause the lead detection system to determine a length of the portion of the section of piping that includes lead content, the length of the portion of piping being calculated as a function of the calculated resistance, the representation of the distance, an inside diameter of the section of piping, an outside diameter of the section of piping.

5. The lead detection system of claim 1, wherein the computer code instructions, when executed by at least one processor, further cause the lead detection system to cause the current source to vary the electrical current flow delivered to the section of piping via the source leads.

6. The lead detection system of claim 5, wherein varying the electrical current flow delivered to the section of piping via the source leads comprises delivering different amounts of electrical current depending on the amount of resistance being measured.

7. The lead detection system of claim 5, wherein varying the electrical current flow delivered to the section of piping via the source leads comprises delivering electrical current flow to the section of piping via the source leads in a first direction, and delivering electrical current flow to the section of piping via the source leads in a second direction.

8. The lead detection system of claim 1, wherein the computer code instructions, when executed by at least one processor, further cause the lead detection system to determine a probability that the determination of whether at least a portion of the section of piping includes lead content is correct.

9. The lead detection system of claim 1, wherein the source leads include a first source lead and a second source lead, the sensor leads include a first sensor lead and a second sensor lead, and wherein the first source lead and the first sensor lead are coupled to locations of the section of piping that are separated by at least 1.5 times a cross-sectional perimeter of the piping, and the second source lead and the second sensor lead are coupled to locations of the section of piping that are separated by at least 1.5 times the cross-sectional perimeter of the piping.

10. A method for detecting a section of lead in piping in a field setting, the method comprising:
coupling source leads of wire to first separated locations of a section of piping of interest in the field setting;
coupling sensor leads of wire to second separated locations of the section of piping of interest, the second separated locations of the section of piping being located between the first separated locations;
delivering electrical current flow to the section of piping via the source leads;
measuring a voltage level between the sensor leads via a voltmeter connected to the sensor leads;
calculating a resistance of the section of piping as a function of the electrical current flow and the voltage level; and
determining whether at least a portion of the section of piping includes lead content as a function of the calculated resistance.

11. The method of claim 10 further including measuring or estimating a distance between the sensor leads; and wherein the determination of whether at least a portion of the section of piping includes lead content is a function of the calculated resistance and the distance between the sensor leads.

12. The method of claim 11 wherein the determination of whether at least a portion of the section of piping includes lead content is a function of the calculated resistance, the distance between the sensor leads, an inside diameter of the section of piping, and an outside diameter of the section piping.

13. The method of claim 11 further including determining a length of the portion of the section of piping that includes lead content, the length of the portion of piping being calculated as a function of the calculated resistance, the distance between the sensor leads, an inside diameter of the section of piping, an outside diameter of the section of piping.

14. The method of claim 10 further including varying the electrical current flow delivered to the section of piping via the source leads.

15. The method of claim 14 wherein varying the electrical current flow delivered to the section of piping via the source leads comprises delivering different amounts of electrical current depending on the amount of resistance being measured.

16. The method of claim 14 wherein varying the electrical current flow delivered to the section of piping via the source leads comprises delivering electrical current flow in a first direction, and delivering electrical current flow in a second direction.

17. The method of claim 16 wherein measuring the voltage level between the sensor leads comprises measuring the voltage level for the first direction of electrical current flow and the second direction of electrical current flow, and averaging resistance for each voltage level.

18. The method of claim 10 wherein determining whether at least a portion of the section of piping includes lead content is a function of the calculated resistance, the measured distance, resistivity of copper, and resistivity of lead.

19. The method of claim 10 further including determining a probability that the determination of whether at least a portion of the section of piping includes lead content is correct.

20. The method of claim 10 wherein the source leads include a first source lead and a second source lead, the sensor leads include a first sensor lead and a second sensor lead, and further including coupling the first source lead and the first sensor lead to locations of the section of piping that are separated by at least 1.5 times a cross-sectional perimeter of the piping, and coupling the second source lead and the second sensor lead to locations of the section of piping that are separated by at least 1.5 times the cross-sectional perimeter of the piping.

21. A method for detecting a section of lead in piping in a field setting, the method comprising:
coupling source leads of wire to first separated locations of a section of piping of interest in the field setting;
coupling sensor leads of wire to second separated locations of the section of piping of interest, the second separated locations of the section of piping being located between the first separated locations;
delivering electrical current flow to the section of piping via the source leads;
measuring a voltage level between the sensor leads via a voltmeter connected to the sensor leads; and
determining whether at least a portion of the section of piping includes lead content as a function of the measured voltage level.
